(19) United States
(12) Patent Application Publication
(10) Pub. No.: US 2024/0204190 A1
(43) Pub. Date: Jun. 20, 2024

(54) DIFFERENTIAL SUSPENDED SINGLE-LAYER GRAPHENE NANOPORE SENSOR, AND PREPARATION METHOD THEREFOR AND USE THEREOF

(71) Applicant: TSINGHUA UNIVERSITY, Beijing (CN)

(72) Inventors: Haidong WANG, Beijing (CN); Siqi XIE, Beijing (CN); Hongxin ZHU, Beijing (CN); Yaohong ZHOU, Beijing (CN); Shuaiyi ZHAO, Beijing (CN)

(21) Appl. No.: 18/590,997

(22) Filed: Feb. 29, 2024

Related U.S. Application Data
(63) Continuation of application No. PCT/CN2023/072678, filed on Jan. 17, 2023.

(30) Foreign Application Priority Data
Apr. 11, 2022 (CN) 202210372933.8

(51) Int. Cl. H01M 4/583; G01N 33/487; H01M 4/36; H01M 4/38

(52) U.S. Cl. CPC H01M 4/583; G01N 33/48721; H01M 4/366; H01M 4/386

(57) ABSTRACT

Provided is a preparation method for a differential suspended single-layer graphene nanopore sensor. The method includes: forming a SiO₂ layer on a silicon substrate layer, and etching a side of the silicon substrate layer facing away from the SiO₂ layer to form a groove; forming a graphene strip unit on the SiO₂ layer, the graphene strip unit including two single-layer grapheme stripes arranged at an interval and stretched across the groove; depositing a metal electrode layer, the electrode layer formed at one side of the groove covering the two single-layer grapheme stripes simultaneously, the electrode layer formed at another side of the groove including two parts arranged at an interval and each covering one of the two single-layer graphene strips; etching away the silicon dioxide layer that is exposed in the region of the groove; and punching nanopores in one of the two single-layer graphene strips suspending.

### CROSS-REFEREMCE TO RELATED APPLICATIONS

This application is a continuation of International Application No. PCT/CN2023/072678 filed on Jan. 17, 2023, which claims priority to Chinese patent application No. 202210372933.8, filed with China National Intellectual Property Administration on Apr. 11, 2022, the entire disclosure of which is incorporated herein by reference.

### FIELD

The present disclosure belongs to the field of single-molecule detection and analysis technology, and more particularly, relates to a differential suspended single-layer graphene nanopore sensor and a preparation method therefor and use thereof.

### BACKGROUND

The development of single-molecule detection and analysis technology plays an important role in promoting disease screening, drug research and development, personalized diagnosis and treatment, biomedical research, and other fields. It can not only be applied to timely virus screening, fetal key gene screening, DNA damage detection, RNA direct sequencing, single-molecule protein sequencing, heavy metal ion detection, and other scenes, but also help people to explore a mystery of human beings from a molecular level, and understand a course of human life.

Among them, DNA sequencing has become a focus of many researchers, and sequencing technology has gradually become mature and commercialized after several generations of reform. The first-generation technology is based on Sanger's dideoxy chain-termination method and Gilbert's chemical cleavage method, using PCR (Polymerase Chain Reaction) amplification combined with fluorescent labeling and capillary array electrophoresis technology to achieve sequencing. This technology has high precision but low throughput and is expensive, with an average sequencing cost of 1 USD per base. The second-generation technology mainly includes Solexa sequencing technology, SOLiD sequencing technology, Complete Genomics sequencing technology, etc. Sequencing while synthesizing based on amplification improves the sequencing throughput and precision while greatly reducing the cost, which has become a mainstream method in a current market. However, the second-generation technologies may only read 100-800 bp (bp: base pair) of DNA fragments in a single run. The third-generation technology mainly includes tSMS single-molecule sequencing technology, SMRT single-molecule real-time technology, VisiGen sequencing technology, etc. Its most important feature is single-molecule sequencing without PCR amplification, with higher throughput and shorter sequencing time. Although a sequencing length is greatly extended (up to 10 kb or more), it is still limited by polymerase activity and photobleaching of fluorophores.

Currently, the latest generation of sequencing technology is nanopore single-molecule sequencing. This kind of technology does not require any chemical pretreatment for single molecules and performs the sequencing by distinguishing changes in electrical signals caused by different base pairs of DNA passing through nanopores. Based on its principle, it is mainly divided into an ionic current detection method, a tunnel current detection method, and a capacitive detection method. Nanopores may be divided into two categories: biological nanopores and solid nanopores. The MinION, launched by Oxford Nanopore Technologies in 2013, is a mature commercial portable biological nanopore sequencer in which an adapter and a motor protein (Enzyme motor) are pre-connected to an end of each to-be-tested DNA fragment, and then a to-be-tested single-stranded DNA molecule and its complementary strand are guided by the adapter to pass through the nanopore in sequence after being unwound by the motor protein, and a change in an ionic current caused by a change in a nucleotide sequence through the pore is detected for DNA sequencing. This instrument currently achieves a raw read accuracy of 99.3%, may obtain up to data of 50 Gb from a single flow cell, and is available for as little as 1,000 USD. Although bio-nanopores have shown excellent detection results and have been commercially applied, they still face several challenges as follows. (1) A biofilm pore embedded in a phospholipid bilayer has a thickness (about 3 nm to 8 nm) much greater than a distance between adjacent bases (0.34 nm), and a measured signal is a result of a combined action of a plurality of bases, affecting a spatial resolution of a single base. (2) The phospholipid bilayer lacks tolerance and stability, which limits a usage count and a service life of the phospholipid bilayer, and meanwhile, it is difficult to integrate the phospholipid bilayer with other devices, which increases a difficulty of scale production. (3) A constant pore diameter of the biological nanopore limits its applicability and generalization ability in other molecular detection fields. In addition, the ionic current detection method currently used in most sequencing devices also has many limitations. (1) An ionic current signal amplitude is not high (only on an order of 1 nA). The accuracy of the single-molecule detection relies heavily on later intelligent signal recognition and data processing, and the reliability and stability of the detection results are thus affected. (2) Single-pore ionic current measurement is prone to pore clogging, leading to failure of a whole device. Although nanopore array measurement can solve the problem of the single-pore clogging and failure while improving the throughput, the array design for ionic currents needs to separate a solution between each nanopore, which greatly increases a processing difficulty and processing cost of the device. (3) Branton et al. reported that in addition to the bases in the nanopores that affect the ionic current signal, it also includes bases in a region where upper and lower sides of the nanopores have thicknesses approximating to diameters of the nanopores. In order to allow the to-be-tested DNA molecule to pass through, the pore diameter must be larger than approximately 1.5 nm, which means that a length of an affected region is at least 3 nm (2×1.5 nm), again much greater than the distance between adjacent bases. To overcome the above-mentioned shortcomings, researchers have developed a solid-state nanopore sensor using silica, alumina, silicon nitride, molybdenum disulfide, and graphene as nanopore film materials. Solid-state nanopores are mechanically strong and chemically and thermally stable, have flexible and adjustable pore diameters, and are easily integrated into other nanodevices, which can be mass-produced using mature semiconductor processes.

To sum up, although scholars in the field of single-molecule sequencing have achieved exciting experimental results and achieved high accuracy, there is still a certain distance from clinical application standards. Faced with the challenge that the detection membrane pores are too thick, it is difficult to highlight the single base signal, and a signal-to-noise ratio of the detection signal needs to be further improved.

### SUMMARY

The present disclosure aims to solve at least one of the technical problems in the related art to some extent. To this end, an object of the present disclosure is to provide a differential suspended single-layer graphene nanopore sensor, a preparation method therefor, and use thereof. The differential suspended single-layer graphene nanopore sensor or the preparation method therefor can obtain an extremely high spatial resolution, can improve signal intensity by detecting a tunnel current, further can improve a signal-to-noise ratio by using a differential circuit. In this way, stability and repeatability of data are greatly improved. Therefore, it is easier to improve sequencing precision.

In one aspect of the present disclosure, the present disclosure provides a preparation method for a differential suspended single-layer graphene nanopore sensor for a single-molecule detection. According to an embodiment of the present disclosure, the method includes:
step 1 of providing a silicon substrate layer with a silicon dioxide layer formed on a surface of a side of the silicon substrate layer;
step 2 of etching a side of the silicon substrate layer facing away from the silicon dioxide layer to form at least one groove at the silicon substrate layer, allowing the silicon dioxide layer located in a region of the at least one groove to suspend;
step 3 of forming a graphene strip unit at a side of the silicon dioxide layer facing away from the silicon substrate layer, the graphene strip unit including two single-layer grapheme stripes arranged at an interval and stretched across the at least one groove;
step 4 of depositing a metal electrode layer on a part of the silicon dioxide layer and the two single-layer graphene strips that are located at two sides of the at least one groove, the metal electrode layer formed at one of the two sides of the at least one groove covering the two single-layer grapheme stripes simultaneously, the metal electrode layer formed at the other one of the two sides of the at least one groove including two parts arranged at an interval, each of the two parts covering one of the two single-layer graphene strips;
step 5 of etching away the silicon dioxide layer that is exposed in the region of the at least one groove by using a hydrofluoric acid solution, allowing the two single-layer graphene strips corresponding to the region of the at least one groove to suspend; and
step 6 of punching nanopores in one of the two single-layer graphene strips suspending in the at least one groove by using an ion beam to obtain the sensor.

The preparation method for the differential suspended single-layer graphene nanopore sensor for the single-molecule detection according to the above embodiment of the present disclosure has at least the following beneficial effects. (1) The method uses the etching method to etch the groove on the silicon substrate, which not only greatly simplifies the design and processing processes of the groove by designing a groove pattern of any shapes based on requirements and controlling the groove dimension, but also simultaneously prepares the plurality of grooves in no communication with each other on the same silicon substrate. In this way, the method is simple in the operation with the high processing precision and the controllable dimension, and the method enables one-piece molding without bonding process and has stronger operability. (2) Using suspended single-layer graphene as the detection material, it is easier to process the nanopores having a smaller nanopore diameter (e.g., below 10 nm, specifically 5 nm, etc.), ensuring that the DNA single-stranded bases can individually and sequentially pass through. (3) The single-layer grapheme has a thickness of only 0.34 nm and is equivalent to a distance between adjacent DNA single-stranded bases, which can greatly improve a spatial resolution of the nanopores. (4) Each groove region may form an independent detection unit, and each detection unit includes two single-layer graphene strips and only one of the two graphene strips has nanopores. As a result, current signals can be collected in two channels, and a signal-to-noise ratio can be improved by using a differential principle, and to greatly improve stability and repeatability of data. (5) A transverse-current-oriented measurement scheme can be used. A tunnel-oriented current signal (100 nA) is significantly greater than a normal ion current signal (1 nA) when a single molecule passes through a pore, and the measurement signal is significantly enhanced. In addition, the same detection unit can complete a two-channel collection of the current signals in same to-be-tested liquid, to allow for more convenient measurement. (6) By controlling the number and distribution of etched grooves and combining the graphene strip units, a sensor having one or more independent detection units can be obtained. A plurality of independent detection units can be arranged in an arrayed distribution or other arrangements, thereby enabling a single sensor test chip to include dozens to hundreds of mutually independent detection units, and through-pore signals of a plurality of molecules can be collected simultaneously. Therefore, even if a certain nanopore is clogged, it will not cause the whole device to fail. Meanwhile, data throughput is significantly improved. (7) The preparation method or the sensor prepared by using the method has wide application prospects in DNA rapid sequencing, RNA direct sequencing, protein single-molecule sequencing, etc.

In addition, the preparation method for the differential suspended single-layer graphene nanopore sensor for the single-molecule detection according to the above embodiment of the present disclosure may further have the following additional technical features.

In some embodiments of the present disclosure, the silicon substrate layer has a plurality of grooves arranged at intervals and in no communication with each other, each of the plurality of grooves is formed as a subunit, the subunit includes two single-layer graphene strips stretched across a same groove of the plurality of grooves and a metal electrode layer deposited on a part of the silicon dioxide layer and two single-layer graphene strips that are located at two sides of the same groove of the plurality of grooves, and single-layer graphene strips between any two of the subunits are in no communication with each other.

In some embodiments of the present disclosure, a spacing between two adjacent subunits ranges from 50 μm to 200 μm; and/or each subunit has a dimension of (300 to 700) μm×(300 to 700) μm.

In some embodiments of the present disclosure, in a same graphene strip unit, a spacing between two single-layer graphene strips ranges from 3 μm to 5 μm.

In some embodiments of the present disclosure, each single-layer graphene strip suspending over one single groove has a suspension length ranging from 500 nm to 1500 nm and a suspension width ranging from 100 nm to 500 nm.

In some embodiments of the present disclosure, each of the nanopores has a pore diameter smaller than or equal to 10 nm.

In some embodiments of the present disclosure, the step 2 includes: step 2-1 of forming a photoresist layer at the side of the silicon substrate layer facing away from the silicon dioxide layer, and developing the photoresist layer into a target shape through electron beam lithography to expose a silicon substrate layer at a etch window; and step 2-2 of etching away the exposed silicon substrate by using xenon difluoride gas to form the at least one groove below the silicon dioxide layer, allowing the silicon dioxide layer located in the region of the at least one groove to suspend.

In some embodiments of the present disclosure, the silicon substrate layer has a thickness ranging from 200 μm to 500 μm.

In some embodiments of the present disclosure, the silicon dioxide layer has a thickness ranging from 100 nm to 300 nm.

In some embodiments of the present disclosure, each of the at least one groove has a groove spacing ranging from 10 μm to 20 μm at a side of the at least one groove close to the silicon dioxide layer.

In some embodiments of the present disclosure, in the step 3, the graphene strip unit is formed on the silicon dioxide layer using photolithography and oxygen plasma.

In some embodiments of the present disclosure, in the step 4, an adhesion layer is deposited on the part of the silicon dioxide layer and the single-layer graphene strip that are located at the two sides of the at least one groove, and the metal electrode layer is deposited on the adhesion layer.

In some embodiments of the present disclosure, the adhesion layer has a thickness ranging from 5 nm to 10 nm; and the metal electrode layer has a thickness ranging from 100 nm to 150 nm.

In some embodiments of the present disclosure, the adhesion layer is a chromium layer; and the metal electrode layer is a gold layer.

In some embodiments of the present disclosure, in the step 6, said punching the nanopores in the one of the suspended single-layer graphene strips in the at least one groove is performed using a focused helium ion beam.

According to a second aspect of the present disclosure, the present disclosure provides a differential suspended single-layer graphene nanopore sensor for a single-molecule detection. According to an embodiment of the present disclosure, this nanopore sensor is prepared by the above-mentioned preparation method for the differential suspended single-layer graphene nanopore sensor for the single-molecule detection. It can be understood that the features and effects described for the above preparation method for the differential suspended single-layer graphene nanopore sensor for the single-molecule detection will apply mutatis mutandis to the differential suspended single-layer graphene nanopore sensor for the single-molecule detection, and details thereof are not repeated herein.

In some embodiments of the present disclosure, the differential suspended single-layer graphene nanopore sensor for the single-molecule detection includes a plurality of sub-sensors. Each of the plurality of sub-sensors includes a subunit. The subunit includes two single-layer graphene strips stretched across a same groove and a metal electrode layer deposited on the part of the silicon dioxide layer and the two single-layer graphene strips that are located at the two sides of the groove.

In some embodiments of the present disclosure, the plurality of sub-sensors is arranged in an array or circumferentially.

According to a third aspect of the present disclosure, the present disclosure provides a single-molecule detection method using the differential suspended single-layer graphene nanopore sensor for the single-molecule detection. Compared with the related art, this method can obtain the extremely high spatial resolution, can improve the signal intensity by detecting the tunnel current, further can improve the signal-to-noise ratio by using the differential circuit, and can further increase detection throughput when the nanopores are highly arrayed. Therefore, an existing nanopore technology bottleneck can be better overcome, and the sequencing precision can be improved to achieve a clinical application standard. It can be understood that features and effects described for the differential suspended single-layer graphene nanopore sensor for the single-molecule detection will apply mutatis mutandis to the single-molecule detection method, and details thereof are not repeated herein.

According to a fourth aspect of the present disclosure, the present disclosure provides use of the above-mentioned differential suspended single-layer graphene nanopore sensor for the single-molecule detection or the above-mentioned single-molecule detection method in DNA sequencing, RNA direct sequencing, and a protein single-molecule detection. Compared with the related art, the differential suspended single-layer graphene nanopore sensor for the single-molecule detection or the single-molecule detection method can obtain the extremely high spatial resolution, can improve the signal intensity by detecting the tunnel current, can further improve the signal-to-noise ratio by using the differential circuit, and can further increase detection throughput when the nanopores are highly arrayed. Therefore, the existing nanopore technology bottleneck can be better overcome, and the sequencing precision can be improved to achieve the clinical application standard. It is of great significance to promote the further development of disease screening, drug research and development, personalized diagnosis and treatment, biomedical research, and other related fields.

Additional aspects and advantages of the embodiments of the present disclosure will be provided at least in part in the following description, or will become apparent in part from the following description, or can be learned from the practice of the embodiments of the present disclosure.

### BRIEF DESCRIPTION OF THE DRAWINGS

These and other aspects and advantages of embodiments of the present disclosure will become apparent and more readily appreciated from the following descriptions made with reference to the accompanying drawings.

FIG. 1 is a flowchart of a preparation method for a differential suspended single-layer graphene nanopore sensor for a single-molecule detection according to an embodiment of the present disclosure.

FIG. 2 is another flowchart of preparation method for a differential suspended single-layer graphene nanopore sensor for a single-molecule detection according to an embodiment of the present disclosure.

FIG. 3 is a schematic structural view of transverse-current-oriented measurement of a differential suspended single-layer graphene nanopore sensor according to an embodiment of the present disclosure.

FIG. 4 is a schematic structural diagram of a 2×2 arrayed differential suspended single-layer graphene nanopore sensing chip according to an embodiment of the present disclosure.

FIG. 5 is an ion beam microscope photograph of graphene nanopores obtained using He⁺ focused ion beams according to an embodiment of the present disclosure.

FIG. 6 is a connection circuit photograph of an array differential suspended single-layer graphene nanopore sensing chip according to an embodiment of the present disclosure.

FIG. 7 is a schematic diagram of improving a signal-to-noise ratio output by a differential according to an embodiment of the present disclosure (where (a) is a current signal of a graphene strip having nanopores, (b) is a current signal of a graphene strip having no nanopore, and (c) is a signal obtained by the differential).

FIG. 8 is a schematic diagram of a signal output result of a multi-detection unit of an array differential suspended single-layer graphene nanopore sensing chip according to an embodiment of the present disclosure.

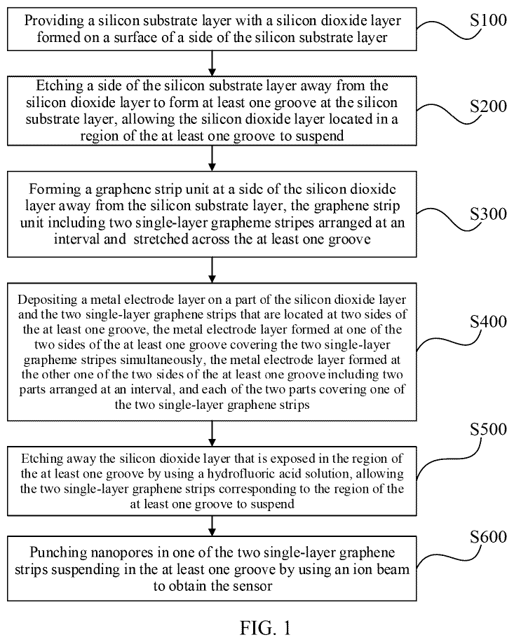

### DETAILED DESCRIPTION

The embodiments of the present disclosure will be described in detail below with reference to examples thereof as illustrated in the accompanying drawings, throughout which same or similar elements, or elements having same or similar functions, are denoted by same or similar reference numerals. The embodiments described below with reference to the drawings are illustrative only, and are intended to explain rather than limit the present disclosure.

In one aspect of the present disclosure, the present disclosure provides a preparation method for a differential suspended single-layer graphene nanopore sensor for a single-molecule detection. According to an embodiment of the present disclosure, the method includes following operations at steps S100 to S600.

At step S100, a silicon substrate layer is provided with a silicon dioxide layer formed on a surface of a side of the silicon substrate layer.

According to embodiments of the present disclosure, thermal oxidation or chemical vapor deposition may be performed on a surface of a side of the silicon substrate to form a SiO₂ layer. A thickness of the SiO₂ layer may be controlled by varying a time for the thermal oxidation of the silicon substrate or by controlling a parameter condition of the chemical vapor deposition.

According to the embodiments of the present disclosure, the silicon substrate and the SiO₂ layer mainly serves a supporting function, and the silicon substrate layer may have a thickness ranging from 200 μm to 500 μm, for example, 250 μm, 300 μm, 350 μm, 400 μm, 450 μm, etc. The SiO₂ layer may have a thickness ranging from 100 nm to 300 nm, for example, 150 nm, 200 nm, 250 nm, etc. Inventors find that if the thickness of the silicon substrate and the SiO₂ layer is too thin, an overall support strength of the device is insufficient, which affects a yield rate and a service life of the device. However, if the thickness of the silicon substrate and the SiO₂ layer is too thick, a subsequent etching depth will be too large, which not only affects etching/preparation efficiency, but also may affect accuracy of dimension control. In the present disclosure, by controlling the silicon substrate and the SiO₂ layer to be within the above-mentioned thickness range, the overall support strength and production efficiency of the device can be better considered, and comprehensive performance such as the yield rate, the production efficiency and the quality and the like of the device can be improved.

At step S200, a side of the silicon substrate layer facing away from the silicon dioxide layer is etched to form at least one groove at the silicon substrate layer, allowing the silicon dioxide layer located in a region of the at least one groove to suspend.

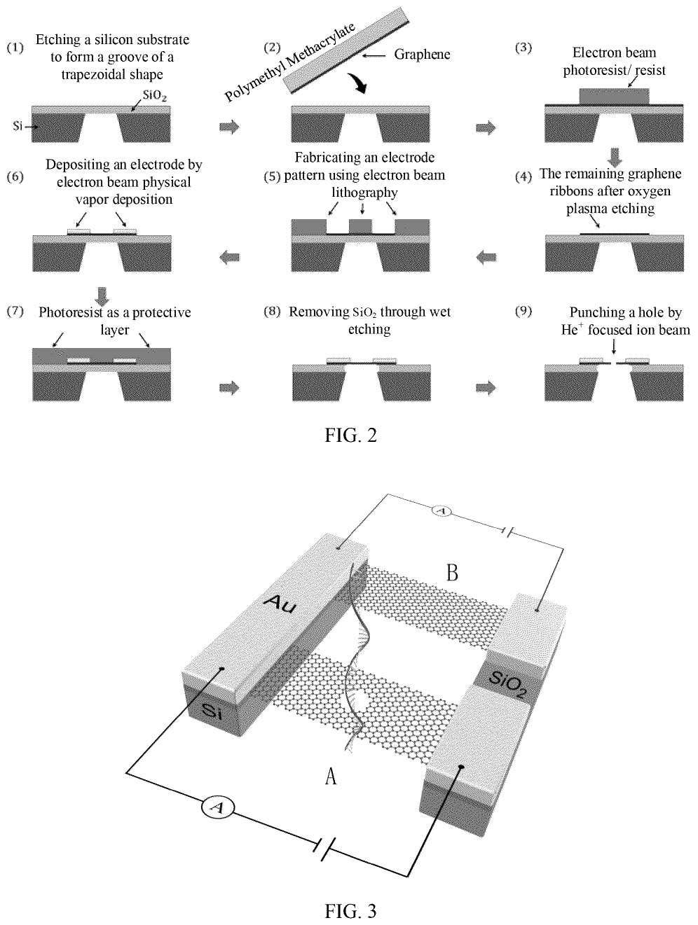

According to the embodiments of the present disclosure, referring to (1) of FIG. 2, a first photoresist layer may be formed on a side of the silicon substrate layer facing away from the SiO₂ layer, and the first photoresist layer may be developed into a target shape through electron beam lithography to expose a silicon substrate layer at an etch window. After that, an obtained product is placed in a xenon difluoride (XeF₂) gas reactor, and the exposed silicon substrate is etched by using the xenon difluoride to form a groove under the SiO₂ layer, and the SiO₂ layer located in a groove region is suspended, such that a product structure as described at (1) of FIG. 2 is obtained. It can be understood that since the silicon substrate at the side facing away from the SiO₂ layer is in contact with the xenon difluoride gas for a relatively long time, the groove obtained by etching has a dimension gradually decreasing as a whole in the direction from the side of the silicon substrate facing away from the SiO₂ layer to the side of the silicon substrate close to the SiO₂ layer.

According to the embodiments of the present disclosure, the groove may have a groove spacing ranging from 10 μm to 20 μm at a side of the groove close to the silicon dioxide layer, i.e., the groove may have a minimum groove spacing ranging from 10 μm to 20 μm, for example, 12 μm, 14 μm, 16 μm or 18 μm, etc. Controlling the groove to be within the above-mentioned dimension range not only satisfies a device dimension requirement required for the single-molecule detection, but also is more beneficial to control a length and width range of the suspended single-layer graphene strip formed in a region corresponding to the groove within a nanometer scale. Thus, a fracture problem caused by overlarge suspended dimension of the single-layer graphene strip can be effectively avoided.

At step S300, a graphene strip unit is formed at the side of the silicon dioxide layer facing away from the silicon substrate layer, the graphene strip unit includes two single-layer grapheme stripes arranged at an interval and simultaneously stretched across the at least one groove.

According to the embodiments of the present disclosure, single-layer graphene may be formed on a copper foil in advance by using a chemical vapor deposition method; a PMMA (Polymethyl Methacrylate) support layer is then formed on the single-layer graphene; and the copper foil is etched with a copper etching solution, and the single-layer graphene is transferred onto the SiO₂ layer using the support layer and the support layer is removed, as shown with reference to (2) of FIG. 2. Further, the graphene strip unit is formed on the silicon dioxide layer using photolithography and oxygen plasma. Referring to (3) of FIG. 2, an electron beam photoresist layer (i.e., a second photoresist layer) with a thickness ranging from 250 nm to 350 nm may be spin-coated on the single-layer graphene, and then electron beam lithography technology is used to expose and develop the second photoresist layer to form a photoresist strip with a submicron width, in which the electron beam photoresist may be ZEP520A (a photoresist layer described below may also be of this type), and a region of the photoresist layer after the electron beam lithography will be dissolved in a developer solution. Further, with reference to (4) of FIG. 2, a product obtained by the electron beam lithography may be exposed to an oxygen plasma environment, and the single-layer graphene not covered by the photoresist will be etched away, thereby forming the graphene strip unit. Inventors find that since the single-layer graphene has an atomic thickness equivalent to a distance between two adjacent bases in a DNA single strand, the detection precision can be greatly improved.

According to the embodiment of the present disclosure, in a same graphene strip unit, a spacing between two single-layer graphene strips ranges from 3 μm to 5 μm, for example, 3.5 μm, 4 μm, 4.5 μm, etc. The two graphene strips in the same graphene strip unit are used to form two independent measurement paths (with reference to FIG. 3), forming a differential circuit. By controlling a spacing between the two single-layer graphene strips to be within the above-mentioned range, a mutual influence of the two measurement paths can be avoided, and it is ensured that the two measurement paths are in a very similar solution measurement environment. Therefore, a more reliable differential effect can be obtained, facilitating improving the measurement precision. Due to reasons such as the flow of the liquid to be measured, the overall offset of the electrical measurement signal may be caused. If detection signals of the differential measurement paths with or without the nanopores are selected for subtraction correction, an adverse effect of an environmental factor on the electrical measurement signal can be eliminated, thereby significantly improving the signal-to-noise ratio.

At step S400, a metal electrode layer is deposited on a part of the silicon dioxide layer and the two single-layer graphene strips that are located at two sides of the at least one groove. The metal electrode layer formed at one of the two sides of the at least one groove covers the two single-layer grapheme stripes simultaneously. The metal electrode layer formed at the other one of the two sides of the at least one groove includes two parts arranged at an interval. Each of the two parts covers one of the two single-layer graphene strips.

According to the embodiments of the present disclosure, a detection unit can be formed by combining a graphene strip unit with a same groove, one detection unit includes two measurement paths forming a differential circuit (as illustrated in FIG. 3), and a single-layer graphene strip corresponding to a same detection unit has a length larger than the groove dimension, including a part corresponding to the groove region and parts located at the opposite sides of the groove (as shown in (4) of FIG. 2). Referring to (5) and (6) of FIG. 2, and FIG. 3, in a process of forming the metal electrode layer, a third photoresist layer may be formed above the single-layer graphene strip and in a region uncovered by the single-layer graphene strip. The third photoresist layer may be formed, using the electron beam lithography, into an exposed pattern matching a target electrode. The metal electrode layer may be formed on the exposed pattern using electron beam physical vapor deposition, and the third photoresist layer may be removed. A part of the formed metal electrode layer is connected to the single-layer graphene strip, and another part is connected to the silicon dioxide layer. As a result, the detection circuit can be obtained, and the single-layer graphene layer can also be fixedly connected to the silicon dioxide layer and the silicon substrate layer.

According to the embodiments of the present disclosure, when the third photoresist layer is formed above the single-layer graphene strip and in the region uncovered by the single-layer graphene strip, the third photoresist layer may be directly formed on the photoresist strip formed in the step S300 and a part of a SiO₂ layer region uncovered by the single-layer graphene strip, or the photoresist strip formed in the step S300 may be removed in advance, and then the third photoresist layer is formed on the single-layer graphene strip and a part of a SiO₂ layer region uncovered by the single-layer graphene strip, as illustrated in (5) of FIG. 2. The third photoresist layer may have a thickness ranging from 250 nm to 350 nm. When the third photoresist layer is directly formed on the single-layer photoresist strip and the part of the SiO₂ layer region uncovered by the single-layer graphene strip, the thickness of the photoresist layer formed in the two regions may be the same or different. Inventors find that the photoresist layer located on the photoresist strip and the SiO₂ layer region uncovered by the single-layer graphene strip does not affect the etching effect even if there is a nanoscale thickness difference in a nanoscale photoresist thickness range.

According to the embodiments of the present disclosure, the metal electrode layer may have a thickness ranging from 100 nm to 150 nm, for example, 100 nm, 110 nm, 120 nm, 140 nm, 150 nm, etc. By controlling the thickness of the electrode layer within the above-mentioned range, it is ensured that the electrode layer has good mechanical strength, conductivity, and bonding strength with the graphene layer, thereby improving the reliability and practicality of the nanopore sensor. Further, the metal electrode layer may preferably be a gold layer, and inventors find that, compared with disadvantages of other metal electrodes that are easily oxidized (such as silver) and have weak adhesion with graphene (such as platinum, which is easily curled when combined with the graphene), a selection of gold as the electrode layer has at least the following advantages: firstly, chemical properties of gold are relatively stable, and ductility is also relatively good; and secondly, an adhesion force of gold is relatively strong.

According to the embodiments of the present disclosure, an adhesion layer is first deposited on the part of the silicon dioxide layer and the single-layer graphene strip that are located at the two sides of the at least one groove, and then the metal electrode layer is deposited on the adhesion layer, which may be realized by the electron beam physical vapor deposition method. Bonding strength between the metal electrode layer and the single-layer graphene strip can be further improved by forming the adhesive layer in advance and then forming the metal electrode layer. In an embodiment, the adhesive layer may be a chromium layer. For the metal electrode layer having the thickness ranging from 100 nm to 150 nm, the adhesive layer may have a thickness ranging from 5 nm to 10 nm. Therefore, mechanical strength and conductivity of the metal electrode layer, as well as its bonding strength with the single-layer graphene strip and the SiO₂ layer can be further ensured to improve the reliability and the practicability of an ultimately fabricated sensor.

At step S500, the silicon dioxide layer that is exposed in the region of the at least one groove is etched away by using a hydrofluoric acid solution, allowing the two single-layer graphene strips corresponding to the region of the at least one groove to suspend.

According to the embodiments of the present disclosure, referring to (7) of FIG. 2, a fourth photoresist layer may be formed on the product prepared in the step S400 in advance to protect the metal electrode layer, the single-layer graphene layer, and the silicon dioxide layer. Then the product is immersed in the hydrofluoric acid solution, and the SiO₂ layer uncovered by the photoresist in the bottom groove will be etched away to form a single-layer graphene layer suspension structure as illustrated in (8) of FIG. 2. In this case, a lattice structure of the graphene will not be affected by the etching process. A concentration of the hydrofluoric acid solution and an etching time used in the process are not particularly limited, and those skilled in the art may select them based on actual needs. For example, a concentration of the hydrofluoric acid solution may range from 3 wt % to 6 wt %, specifically 3 wt %, 3.5 wt %, 4 wt %, 4.5 wt %, 5 wt %, 5.5 wt %, 6 wt %, etc., and an etching time may range from 1 minute to 60 minutes, specifically 5 minutes, 10 minutes, 15 minutes, 30 minutes, etc.

According to the embodiments of the present disclosure, each single-layer graphene strip suspended over the single groove may have a suspension length ranging from 500 nm to 1500 nm, for example, 600 nm, 800 nm, 1000 nm, 1200 nm, 1400 nm, etc. Each single-layer graphene strip suspended over the single groove may have a suspension width ranging from 100 nm to 500 nm, for example, 150 nm, 200 nm, 250 nm, 300 nm, 350 nm, 400 nm, 450 nm, etc. The suspension width of each single-layer graphene strip may be obtained by controlling the width of the single-layer graphene strip, and the suspension length of each single-layer graphene strip may be obtained by controlling a spacing between metal electrode layers located at two sides of the single-layer graphene strip. Inventors find that if a suspension dimension of the single-layer graphene strip is too large, the single-layer graphene strip is easily broken. By controlling the suspension dimension of the single-layer graphene strip within the above-mentioned range, it is more advantageous to ensure the yield rate and service performance of the ultimately prepared sensor are more favorably ensured.

At step S600, nanopores are punched in one of the two single-layer graphene strips suspending in the at least one groove by using an ion beam to obtain the sensor.

According to the embodiments of the present disclosure, referring to (9) of FIG. 2 and FIG. 3, different ions may be used to perforate one of the two single-layer graphene strips suspending in the same groove. In order to allow an edge of the nanopore to be more regular and a diameter of the nanopore as small as possible to improve sensitivity thereof, the ion may preferably be a helium ion having a smaller molecular weight. The perforated device can be understood with reference to a three-dimensional structure of FIG. 3. It should be noted that, in a three-dimensional structure view illustrated in FIG. 3, silicon substrate layers in an A direction and a B direction are actually in communication with each other without a spacing, and the silicon substrate layer located below the two single-layer graphene strips may form a closed space, which can be understood with reference to a plan view illustrated in FIG. 4.

According to the embodiments of the present disclosure, each of the nanopores may have a pore diameter smaller than or equal to 10 nm, for example, may range from 5 nm to 10 nm, and the pore diameter of the nanopore may be specifically controlled based on a dimension of the to-be-tested molecule. Nanopores having a minimum pore diameter of 5 nm may be obtained by using a focused helium ion beam to punch the nanopores on one of the single-layer graphene strips suspending in the groove, which is more conducive to improving precision of DNA molecular fragment sequencing.

According to the embodiments of the present disclosure, with reference to FIG. 4, the silicon substrate layer has a plurality of grooves arranged at intervals and in no communication with each other, each of the plurality of grooves is formed as a subunit, the subunit includes two single-layer graphene strips stretched across a same groove of the plurality of grooves and a metal electrode layer deposited on a part of the silicon dioxide layer and two single-layer graphene strips that are located at two sides of the same groove of the plurality of grooves, and single-layer graphene strips between any two of the subunits are in no communication with each other. Therefore, the plurality of subunits may be formed on the silicon substrate, and each of the plurality of subunits may be finally formed as an independent detection unit to form a sensor structure in which the plurality of mutually independent detection units may be arranged in a ring or an array (each detection unit is equivalent to an independent sub-sensor, and FIG. 4 shows a sensor structure arranged in a 2×2 array). In an embodiment, several, dozens or hundreds of mutually independent detection units may be formed on a silicon substrate. Thus, through-pore signals of a plurality of molecules can be collected simultaneously, greatly improving the data throughput, and even if one or two nanopores are clogged, test precision of the whole device is not affected much.

According to the embodiment of the present disclosure, with reference to FIG. 4, a spacing between two adjacent subunits may range from 50 μm to 200 μm, for example, 100 μm, 150 μm, etc., and each subunit may have a dimension of (300 to 700) μm×(300 to 700) μm, for example, 500 μm x 500 μm, etc. By controlling the subunit within the above dimension range, test requirements can be met to form the plurality of mutually independent detection units in the same silicon substrate. Furthermore, only one drop of to-be-tested liquid is needed to rapidly enter the plurality of independent detection units to realize simultaneous detection. Therefore, it is convenient to operation, obtaining the extremely high spatial resolution, improving the signal intensity by detecting the tunnel current, reducing the signal-to-noise ratio by using the differential circuit. Further, the detection throughput is increased by highly arrayed nanopores, overcoming the existing nanopore technology bottleneck. The sequencing precision can be improved through a plurality of readings to reach the clinical application standard, which is more conducive to promoting the further development of disease screening, the drug research and development, the personalized diagnosis and treatment, the biomedical research, and other related fields.

To sum up, the preparation method for the differential suspended single-layer graphene nanopore sensor for the single-molecule detection according to the above-mentioned embodiments of the present disclosure has at least the following beneficial effects. (1) The method uses the etching method to etch the groove on the silicon substrate, which not only greatly simplify the design and processing processes of the groove by designing the groove pattern of any shapes based on requirements and controlling the groove dimension, but also simultaneously prepares the plurality of grooves in no communication with each other on the same silicon substrate. In this way, the method is simple in the operation with the high processing precision and the controllable dimension, and the method enables one-piece molding without bonding process and has stronger operability. (2) Using suspended single-layer graphene as the detection material, it is easier to process the nanopores having the smaller nanopore diameter (e.g., below 10 nm, specifically 5 nm, etc.), ensuring that the DNA single-stranded bases can individually and sequentially pass through. (3) The single-layer grapheme has a thickness of only 0.34 nm and is equivalent to a distance between adjacent DNA single-stranded bases, which can greatly improve the spatial resolution of the nanopores. (4) Each groove region may form an independent detection unit, and each detection unit includes two single-layer graphene strips and only one of the two graphene strips has nanopores. As a result, current signals can be collected in two channels, and a signal-to-noise ratio can be improved by using a differential principle, and to greatly improve stability and repeatability of data. (5) A transverse-current-oriented measurement scheme can be used. A tunnel-oriented current signal (100 nA) is significantly greater than a normal ionic current signal (1 nA) when a single molecule passes through a pore, and the measurement signal is significantly enhanced. In addition, the same detection unit can complete a two-channel collection of the current signals in a same to-be-tested liquid, to allow for more convenient measurement. (6) By controlling the number and distribution of etched grooves and combining the graphene strip units, a sensor having one or more independent detection units can be obtained. A plurality of independent detection units can be arranged in an arrayed distribution or other arrangement, thereby enabling a single sensor test chip to include dozens to hundreds of mutually independent detection units, and through-pore signals of a plurality of molecules can be collected simultaneously. Therefore, even if a certain nanopore is clogged, it will not cause the whole device to fail. Meanwhile, data throughput is significantly improved. (7) The preparation method or the sensor prepared by using the method has wide application prospects in DNA rapid sequencing, RNA direct sequencing, protein single-molecule sequencing, etc.

According to a second aspect of the present disclosure, the present disclosure provides a differential suspended single-layer graphene nanopore sensor for a single-molecule detection. According to an embodiment of the present disclosure, this nanopore sensor is prepared by using the above-mentioned preparation method for the differential suspended single-layer graphene nanopore sensor for the single-molecule detection. It can be understood that the features and effects described for the above preparation method for the differential suspended single-layer graphene nanopore sensor for the single-molecule detection will apply mutatis mutandis to the differential suspended single-layer graphene nanopore sensor for the single-molecule detection, and details thereof are not repeated herein.

According to embodiments of the present disclosure, with reference to FIG. 4, the differential suspended single-layer graphene nanopore sensor for the single molecule detection includes a plurality of sub-sensors. Each of the plurality of sub-sensors includes a subunit. The subunit includes two single-layer graphene strips stretched across the same groove and a metal electrode layer deposited on the part of the silicon dioxide layer and the two single-layer graphene strips that are located at the two sides of the groove. The plurality of sub-sensors is arranged in an array or circumferentially, and the plurality of sub-sensors are independent of each other and do not affect each other. Thus, through-pore signals of a plurality of molecules can be collected simultaneously, greatly improving the data throughput, and even if one or two nanopores are clogged, test precision of the whole device is not affected much.

According to a third aspect of the present disclosure, the present disclosure provides a single-molecule detection method for the differential suspended single-layer graphene nanopore sensor for the single-molecule detection. Compared with the related art, this method can obtain the extremely high spatial resolution, can improve the signal intensity by detecting the tunnel current, further improves the signal-to-noise ratio by using the differential circuit, and can further increase detection throughput when the nanopores are highly arrayed. Therefore, an existing nanopore technology bottleneck can be better overcome, and the sequencing precision can be improved to achieve a clinical application standard. It can be understood that features and effects described for the differential suspended single-layer graphene nanopore sensor for the single-molecule detection will apply mutatis mutandis to the single-molecule detection method, and details thereof are not repeated herein.

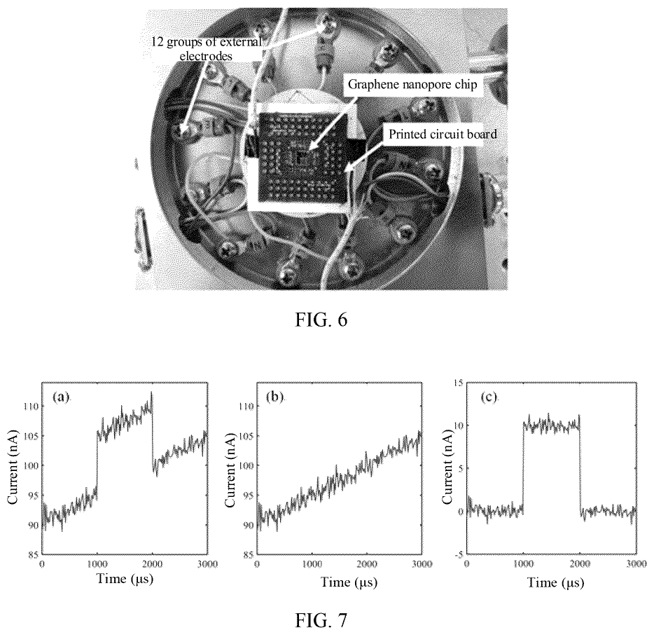

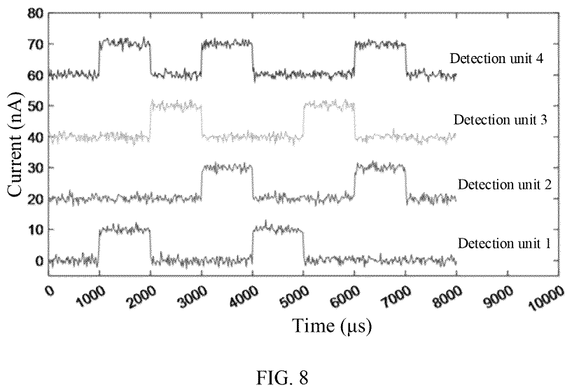

According to a specific example of the present disclosure, the above-mentioned single-molecule detection method may specifically include the followings. (i) The differential suspended single-layer graphene nanopore sensor for the single-molecule detection is in communication with an external circuit through a gold electrode to form an electrical measurement system. Specifically, the gold electrode of the sensor may be connected to a printed circuit board, and then connected to an ultra-low noise current amplifier, a multi-channel data acquisition system, etc. to transmit the measurement signals to an engineering computer. The printed circuit board and the external electrode used are illustrated in FIG. 6. (ii) An electrolyte solution containing to-be-tested molecules is dropped on the sensor having the nanopores, and a voltage is applied to the sensor in a normal direction of the sensor to drive the to-be-tested molecules through the nanopores. (iii) An in-plane current of the single-layer graphene nanopores is measured by using a lapped electrical measurement system. A single detection unit is configured to measure currents passing through two graphene strips with and without pores, respectively (as illustrated in FIG. 3). An expected measurement result of a channel with the nanopores is illustrated in (a) of FIG. 7, and an expected measurement result of a channel without the nanopores is illustrated in (b) of FIG. 7. An expected ultimate output result of the detection unit is obtained by subtracting the two results (as illustrated in (c) of FIG. 7). (iv) FIG. 8 illustrates expected measurement results of a plurality of detection units after differentiation, and a sequencing result may be obtained by analyzing the measurement results. Therefore, the extremely high spatial resolution can be obtained, the signal intensity can be improved, and the signal-to-noise ratio can be further improved. Meanwhile, the detection throughput can be further increased to overcome the existing nanopore technology bottleneck, and the sequencing precision can be improved to achieve the clinical application standard.

According to a fourth aspect of the present disclosure, the present disclosure provides use of the above-mentioned differential suspended single-layer graphene nanopore sensor for the single-molecule detection or the above-mentioned single-molecule detection method in DNA sequencing, RNA direct sequencing and a protein single-molecule detection. Compared with the related art, the differential suspended single-layer graphene nanopore sensor for the single-molecule detection or the single-molecule detection method can obtain the extremely high spatial resolution, can improve the signal intensity by detecting the tunnel current, further improves the signal-to-noise ratio by using the differential circuit, and can further increase detection throughput when the nanopores are highly arrayed. Therefore, the existing nanopore technology bottleneck can be better overcome, and the sequencing precision can be improved to achieve the clinical application standard. It is of great significance to promote the further development of disease screening, drug research and development, personalized diagnosis and treatment, biomedical research, and other related fields. It should be noted that the differential suspended single-layer graphene nanopore sensor for the single-molecule detection and the single-molecule detection method are also suitable for this application, and details thereof will not be repeated herein.

Embodiments of the present disclosure are described in detail below. The embodiments described below are exemplary and are used only to explain the present disclosure and are not to be construed as a limitation of the present disclosure. Where no specific techniques or conditions are indicated in the embodiments, techniques or conditions described in the literature of the field or in accordance with the product specification should be applied. Reagents or instruments that are not specified by the manufacturer are commonly available products that can be obtained through commercial purchase.

### First Embodiment

(1) Preparation of an arrayed differential suspended single-layer graphene nanopore sensor for a single-molecule detection.

(1-1) A silicon dioxide layer having a thickness of 300 nm is deposited on a surface of a silicon substrate having a length and a width of 15 mm×15 mm and a thickness of 500 μm by a chemical vapor deposition method.

(1-2) An electron beam photoresist (ZEP520A) having a thickness of 300 nm is spin-coated on a surface of a side of the silicon substrate facing away from the SiO₂ layer, the photoresist is exposed and developed to form four etch windows through electron beam lithography.

(1-3) The substrate obtained in the step (1-2) is placed in a Xenon difluoride (XeF₂) gas reactor, so the silicon substrate is etched to form four grooves, allowing the SiO₂ layer to partially suspend (as illustrated in (1) of FIG. 2).

(1-4) Single-layer graphene (SLG) is prepared by a chemical vapor deposition method and then the single-layer graphene is transferred to the SiO₂ layer of a chip (as illustrated in (2) of FIG. 2).

(1-4) The electron beam photoresist having the thickness of 300 nm is spin-coated on a surface of the single-layer graphene, the photoresist is exposed and developed through the electron beam lithography to form a photoresist stripe array having a submicron width (as illustrated in (3) of FIG. 2).

(1-5) A product obtained in the step (1-4) is exposed to O₂ (oxygen) plasma for 30 seconds to etch away the single-layer graphene uncovered by the photoresist (as illustrated in (4) of FIG. 2).

(1-6) Another layer of photoresist having a thickness of 300 nm is spin-coated on a top of the remaining single-layer graphene, and a pattern matching a target electrode array structure is formed through the electron beam lithography (as illustrated in (5) of FIG. 2).

(1-7) A Cr adhesion layer having a thickness of 8 nm and an Au thin membrane having a thickness of 120 nm are sequentially deposited on a predetermined region matching the target electrode array structure by using an electron beam physical vapor deposition method, and then an obtained product is immersed in a dimethylacetamide (ZDMAC) solution at a temperature of 45° C. for 10 minutes to remove the photoresist and the Au layer deposited on the photoresist in a non-electrode array region (as illustrated in (6) of FIG. 2).

(1-8) Yet another layer of photoresist having a thickness of 300 nm is spin-coated on an electrode and the SiO₂ uncovered by the SLG as a protective layer (as illustrated in (7) of FIG. 2).

(1-9) The chip is rinsed in deionized water and is transferred to 37% hydrofluoric acid buffer solution for 5-minute immersion to remove the SiO₂ layer below the single-layer graphene. Then, the chip is transferred to the deionized water and ethanol is dropped into the water at a very slow rate until its concentration reaches about 90% (this process takes about 12 hours). After that, the chip is carefully transferred into 100% ethanol and 100% acetone. As last, the chip is dried through supercritical point drying (SCPD) to obtain a suspend single-layer graphene having electrodes at two ends thereof (as illustrated in (8) of FIG. 2).

(1-10) A nanopore having a diameter of 5 nm is punched in a center of a window where one of the single-layer graphene strips is exposed in each detection unit of the chip by using a focused helium ion beam (as illustrated in (9) of FIG. 2).

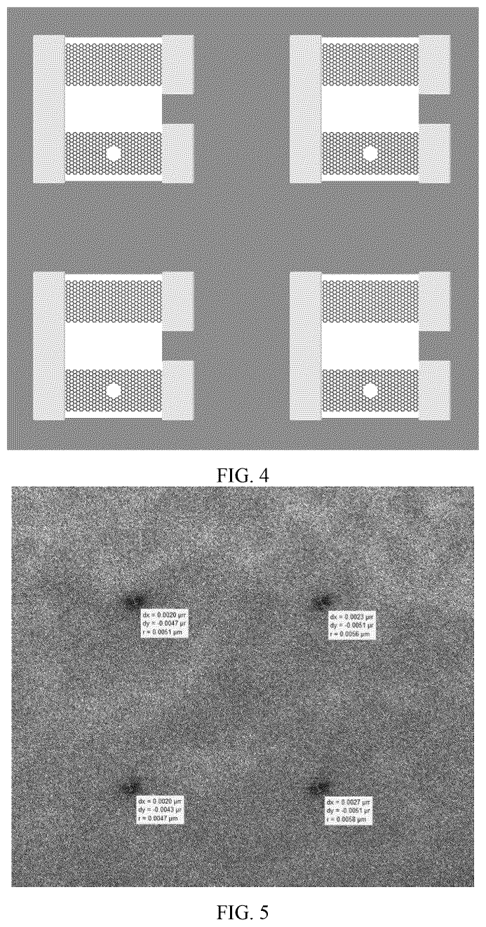

In this embodiment, Zeiss Helium-Neon-Gallium focused ion beam microscope is used to perforate the graphene by the He+ focused ion beam, and a perforation effect is illustrated in FIG. 5. After the perforation is completed, a physical picture of an array differential structure is illustrated in FIG. 4 as a graphene nanopore chip.

(2) A single-molecule detection test

(2-1) A gold electrode of the obtained product in the step (1-10) is connected to a printed circuit board, and then connected to an ultra-low noise current amplifier, a multi-channel data acquisition system, etc. to transmit measurement signals to an engineering computer. The printed circuit board and the external electrode used are illustrated in FIG. 6.

(2-2) An electrolyte solution containing to-be-tested molecules is dropped on a nanopore sensor chip, and a voltage is applied in a normal direction of the sensor to drive the to-be-tested molecules through the nanopores.

(2-3) An in-plane current of the single-layer graphene nanopores is measured by using a lapped electrical measurement system. A single detection unit is configured to measure currents passing through two graphene strips with and without pores, respectively (as illustrated in FIG. 3 or FIG. 4). An expected measurement result of a channel with the nanopores is illustrated in (a) of FIG. 7, and an expected measurement result of a channel without the nanopores is illustrated in (b) of FIG. 7. An expected ultimate output result of the detection unit is obtained by subtracting the two results (as illustrated in (c) of FIG. 7).

(2-4) FIG. 8 illustrates expected measurement results of a plurality of detection units after differentiation, and a sequencing result can be obtained by analyzing the measurement results.

In the description of the present disclosure, it is to be understood that, terms such as “length”, “width”, “thickness”, “over”, “below”, “in”, “out”, etc., refer to the directions and location relations which are the directions and location relations shown in the drawings, and for describing the present disclosure and for describing in simple, and which are not intended to indicate or imply that the device or the elements are disposed to locate at the specific directions or are structured and performed in the specific directions, which could not to be understood to the limitation of the present disclosure. In addition, terms such as “first” and “second” are used herein for purposes of description and are not intended to indicate or imply relative importance or significance. Furthermore, the feature defined with “first” and “second” may include one or more this feature distinctly or implicitly. In the description of the present disclosure, “a plurality of” means two or more than two, unless specified otherwise. In the present disclosure, unless specified or limited otherwise, the terms “mounted,” “connected,” “coupled” and “fixed” are understood broadly, such as fixed, detachable mountings, connections and couplings or integrated, and can be mechanical or electrical mountings, connections and couplings, and also can be direct and via media indirect mountings, connections, and couplings, and further can be inner mountings, connections and couplings of two components or interaction relations between two components, which can be understood by those skilled in the art according to the detail embodiment of the present disclosure. In the present disclosure, unless specified or limited otherwise, the first characteristic is “on” or “under” the second characteristic refers to the first characteristic and the second characteristic can be direct or via media indirect mountings, connections, and couplings. And, the first characteristic is “on”, “above”, “over” the second characteristic may refer to the first characteristic is right over the second characteristic or is diagonal above the second characteristic, or just refer to the horizontal height of the first characteristic is higher than the horizontal height of the second characteristic. The first characteristic is “below” or “under” the second characteristic may refer to the first characteristic is right over the second characteristic or is diagonal under the second characteristic, or just refer to the horizontal height of the first characteristic is lower than the horizontal height of the second characteristic.

In the description of the present disclosure, reference throughout this specification to “an embodiment”, “some embodiments”, “an example”, “a specific example”, or “some examples” means that a particular feature, structure, material, or characteristic described in connection with the embodiment or example is included in at least one embodiment or example of the present disclosure. Thus, the appearances of the phrases in various places throughout this specification are not necessarily referring to the same embodiment or example of the present disclosure. Furthermore, the particular features, structures, materials, or characteristics may be combined in any suitable manner in one or more embodiments or examples. Without a contradiction, the different embodiments or examples and the features of the different embodiments or examples can be combined by those skilled in the art.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

What is claimed is:

1. A preparation method for a differential suspended single-layer graphene nanopore sensor for a single-molecule detection, comprising:
step 1 of providing a silicon substrate layer with a silicon dioxide layer formed on a surface of a side of the silicon substrate layer;
step 2 of etching a side of the silicon substrate layer facing away from the silicon dioxide layer to form at least one groove at the silicon substrate layer, allowing the silicon dioxide layer located in a region of the at least one groove to suspend;
step 3 of forming a graphene strip unit at a side of the silicon dioxide layer facing away from the silicon substrate layer, the graphene strip unit comprising two single-layer grapheme stripes arranged at an interval and stretched across the at least one groove;
step 4 of depositing a metal electrode layer on a part of the silicon dioxide layer and the two single-layer graphene strips that are located at two sides of the at least one groove, the metal electrode layer formed at one of the two sides of the at least one groove covering the two single-layer grapheme stripes simultaneously, the metal electrode layer formed at the other one of the two sides of the at least one groove comprising two parts arranged at an interval, and each of the two parts covering one of the two single-layer graphene strips;
step 5 of etching away the silicon dioxide layer that is exposed in the region of the at least one groove by using a hydrofluoric acid solution, allowing the two single-layer graphene strips corresponding to the region of the at least one groove to suspend; and
step 6 of punching nanopores in one of the two single-layer graphene strips suspending in the at least one groove by using an ion beam to obtain the sensor.

2. The preparation method according to claim 1, wherein the silicon substrate layer has a plurality of grooves arranged at intervals and in no communication with each other, each of the plurality of grooves being formed as a subunit, the subunit comprising two single-layer graphene strips stretched across a same groove of the plurality of grooves and a metal electrode layer deposited on a part of the silicon dioxide layer and two single-layer graphene strips that are located at two sides of the same groove of the plurality of grooves, and single-layer graphene strips between any two of the subunits being in no communication with each other.

3. The preparation method according to claim 2, wherein:
a spacing between two adjacent subunits ranges from 50 μm to 200 μm; and/or
each subunit has a dimension of (300 to 700) μm×(300 to 700) μm.

4. The preparation method according to claim 1, satisfying at least one of the following conditions:
in a same graphene strip unit, a spacing between two single-layer graphene strips ranges from 3 μm to 5 μm;
each single-layer graphene strip suspending over one single groove has a suspension length ranging from 500 nm to 1500 nm and a suspension width ranging from 100 nm to 500 nm; and
each of the nanopores has a pore diameter smaller than or equal to 10 nm.

5. The preparation method according to claim 1, wherein the step 2 comprises:
step 2-1 of forming a photoresist layer at the side of the silicon substrate layer facing away from the silicon dioxide layer, and developing the photoresist layer into a target shape through electron beam lithography to expose a silicon substrate layer at an etch window; and
step 2-2 of etching away the exposed silicon substrate by using xenon difluoride gas to form the at least one groove below the silicon dioxide layer, allowing the silicon dioxide layer located in the region of the at least one groove to suspend.

6. The preparation method according to claim 1, wherein the silicon substrate layer has a thickness ranging from 200 μm to 500 μm.

7. The preparation method according to claim 1, wherein the silicon dioxide layer has a thickness ranging from 100 μm to 300 nm.

8. The preparation method according to claim 1, wherein each of the at least one groove has a groove spacing ranging from 10 μm to 20 μm at a side of the at least one groove close to the silicon dioxide layer.

9. The preparation method according to claim 1, wherein in the step 3, the graphene strip unit is formed on the silicon dioxide layer using photolithography and oxygen plasma.

10. The preparation method according to claim 1, wherein in the step 4, an adhesion layer is deposited on the part of the silicon dioxide layer and the single-layer graphene strip that are located at the two sides of the at least one groove, and the metal electrode layer is deposited on the adhesion layer.

11. The preparation method according to claim 10, wherein:
the adhesion layer has a thickness ranging from 5 nm to 10 nm; and
the metal electrode layer has a thickness ranging from 100 nm to 150 nm.

12. The preparation method according to claim 10, wherein:
the adhesion layer is a chromium layer; and
the metal electrode layer is a gold layer.

13. The preparation method according to claim 1, wherein in the step 6, said punching the nanopores in the one of the suspended single-layer graphene strips in the at least one groove is performed using a focused helium ion beam.

14. A differential suspended single-layer graphene nanopore sensor for a single-molecule detection prepared by the preparation method according to claim 1.

15. The sensor according to claim 14, comprising a plurality of sub-sensors, each of the plurality of sub-sensors comprising a subunit, the subunit comprising two single-layer graphene strips stretched across a same groove and a metal electrode layer deposited on the part of the silicon dioxide layer and the two single-layer graphene strips that are located at the two sides of the same groove.

16. The sensor according to claim 15, wherein the plurality of sub-sensors is arranged in an array or circumferentially.

17. A single-molecule detection method using the differential suspended single-layer graphene nanopore sensor for the single-molecule detection according to claim 14.

18. Use of the differential suspended single-layer graphene nanopore sensor for the single-molecule detection according to claim 14 in DNA sequencing, direct RNA sequencing, and a protein single-molecule detection.

19. Use of the single-molecule detection method according to claim 17 in DNA sequencing, direct RNA sequencing, and a protein single-molecule detection.
